(19) United States
(12) Patent Application Publication
(10) Pub. No.: US 2025/0228476 A1
(43) Pub. Date: Jul. 17, 2025

(54) PHYSIOLOGICAL VALUE SENSING DEVICE AND SENSING METHOD THEREOF

(71) Applicant: Taiwan-Asia Semiconductor Corporation, Hsinchu City (TW)

(72) Inventor: Chun-Yen YU, Hsinchu City (TW)

(21) Appl. No.: 18/642,261

(22) Filed: Apr. 22, 2024

(30) Foreign Application Priority Data
Jan. 11, 2024 (TW) 113101177

(51) Int. Cl. A61B 5/1455; A61B 5/145

(52) U.S. Cl. CPC A61B 5/1455; A61B 5/14532; A61B 5/14546; A61B 2562/0238; A61B 2562/0242; A61B 2562/046

(57) ABSTRACT

A physiological value sensing device and a sensing method thereof are provided. The physiological value sensing device comprises an input module and a computing module. The input module is used to provide a multi-band light to illuminate a testee to generate a plurality of optical signals, and the optical signals include a plurality of interference signals and a physiological target signal. The computing module is used to establish a physiological normal model. The physiological normal model has multiple physiological sample values corresponding to the multi-band light. The computing module converts the interference signals and the physiological target signal into the physiological normal model for fitting. A physiological target value corresponding to the physiological target signal is generated after eliminating the interference signals based on the physiological sample values.

### CROSS-REFERENCES TO RELATED APPLICATIONS

This application claims the benefit of priority to Taiwanese Patent Application No. 113101177 filed on Jan. 11, 2024, which is hereby incorporated by reference in its entirety.

### BACKGROUND OF THE INVENTION

### Field of the Invention

The present invention relates to a physiological value sensing device and a sensing method thereof, and in particular to a non-invasive blood glucose sensing device and a sensing method thereof.

### Descriptions of the Related Art

Non-invasive blood glucose testing technology is a technology that does not require puncturing the skin or extracting blood samples to measure blood glucose levels. The most obvious advantage of this type of technology is that it does not require the use of needles for puncture, which is more efficient, more comfortable and reduces patient pain and discomfort than traditional blood glucose testing methods. In addition, non-invasive glucose testing methods also reduce the risk of infection for testees so that the public health will be promoted positively. Moreover, the operation of such devices is simple and convenient for users to operate at home or in daily life without requiring special training or the assistance of medical professionals. Most importantly, non-invasive glucose testing methods can provide continuous monitoring capabilities and provide more detailed data at all times for helping patients to manage related diseases more easily.

However, non-invasive glucose detection technology still has some shortcomings. For example, the accuracy of current non-invasive blood glucose testing technology is relatively low and is easily affected by a variety of factors, including poor optical performance of the testing equipment, individual differences in testees, and interference from various physiological values in the testees' bodies. The accuracy of non-invasive blood glucose testing technology is often questioned accordingly. In order to overcome the above problems, the industry urgently needs an innovative non-invasive physiological value sensing device and its sensing method to solve the above problems.

### SUMMARY OF THE INVENTION

The main objective of the present invention is to provide an innovative physiological value sensing device and a sensing method thereof to solve the problems, such as, poor optical performance of the conventional technology, the individual differences of testees, and the interference of various physiological values in the testees' bodies which will affect sensing accuracy.

To achieve the above objective, the present invention discloses a physiological value sensing device which comprises an input module and a computing module. The input module is used to provide a multi-band light to illuminate a testee to generate a plurality of optical signals. The optical signals include a plurality of interference signals and a physiological target signal. The computing module is used to create a physiological normal model. The physiological normal model has multiple physiological sample values corresponding to the multi-band light. The computing module converts the interference signals and the physiological target signal into the physiological normal model for fitting. A physiological target value corresponding to the physiological target signal is generated after eliminating the interference signals based on the physiological sample values.

In one embodiment of the physiological value sensing device of the present invention, the input module includes a plurality of light emitting units and at least one light detection unit, wherein the light emitting units are used to provide the multi-band light and are capable of adjusting the light intensity of the multi-band light and the at least one light detection unit is used to receive a reflection light reflected by the multi-band light illuminating the testee and generate the optical signals corresponding to the reflection light.

In one embodiment of the physiological value sensing device of the present invention, the light emitting units are capable of providing the multi-band light with a wavelength of 800 nanometers to 1700 nanometers and a wavelength interval of not less than 100 nanometers.

In one embodiment of the physiological value sensing device of the present invention, the input module further includes a microstructure, disposed adjacent to the at least one light detection unit to prevent the multi-band light provided by the light emitting units from being received by the at least one light detection unit without being reflected by the testee.

In one embodiment of the physiological value sensing device of the present invention, the computing module includes a processing sub-module, a fitting sub-module and an evaluation sub-module, wherein the processing sub-module is used to process and convert the interference signals and the physiological target signal into the physiological normal model, the fitting sub-module is used to fit the converted interference signals and the converted physiological target signal with the physiological normal model to generate a fitting result, and eliminate the converted interference signals based on the physiological sample values and generate the physiological target value corresponding to the converted physiological target signal, and the evaluation sub-module is used to output a feature importance evaluation corresponding to the fitting result.

In one embodiment of the physiological value sensing device of the present invention, the physiological value sensing device further comprises a storage module for storing the optical signals generated by the input module, the converted interference signals and the converted physiological target signal, the physiological target value and the feature importance evaluation.

In one embodiment of the physiological value sensing device of the present invention, the physiological value sensing device of further comprises a post processing module for creating a long-term trend report corresponding to the optical signals.

In one embodiment of the physiological value sensing device of the present invention, the physiological value sensing device further comprises an output module for outputting the physiological target value and the feature importance evaluation and the long-term trend report.

To achieve the above objective, the present invention discloses a physiological value sensing method which comprises the following steps: creating a physiological normal model wherein the physiological normal model has multiple physiological sample values corresponding to a multi-band light; providing the multi-band light to illuminate a testee to generate a plurality of optical signals wherein the optical signals include a plurality of interference signals and a physiological target signal; converting the interference signals and the physiological target signal into the physiological normal model for fitting; eliminating the interference signals based on the physiological sample values; and generating a physiological target value corresponding to the physiological target signal.

In one embodiment of the physiological value sensing method of the present invention, the step of providing the multi-band light is to provide the multi-band light with a wavelength of 800 nanometers to 1700 nanometers and a wavelength interval of not less than 100 nanometers.

In one embodiment of the physiological value sensing method of the present invention, the physiological value sensing method further comprises a step of providing an artificial intelligence model for fitting the converted interference signals and the converted physiological target signal with the physiological sample values of the physiological normal model to generate a fitting result, and generating the physiological target value corresponding to the converted physiological target signal after eliminate the converted interference signals based on the fitting result.

After referring to the drawings and the embodiments as described in the following, those the ordinary skilled in this art can understand other objectives of the present invention, as well as the technical means and embodiments of the present invention.

### BRIEF DESCRIPTION OF THE DRAWINGS

FIG. 1 depicts a schematic diagram illustrating the relative infrared light absorption rate variation of various substances in human tissues;

FIG. 2 depicts a schematic diagram of the physiological value sensing device of the present invention;

FIG. 3 depicts a top view schematic diagram of several embodiments of the input module in the physiological value sensing device of the present invention;

FIG. 4 depicts a schematic diagram of the fitting sub-module performing model fitting in an embodiment of the present invention;

FIG. 5 depicts a schematic diagram of the decision tree of the decision making history in an embodiment of the present invention; and

FIG. 6 depicts a flowchart illustrating the process of the physiological value sensing method of the present invention.

### DETAILED DESCRIPTION OF THE PREFERRED EMBODIMENT

In the following description, the present invention will be explained with reference to various embodiments thereof. These embodiments of the present invention are not intended to limit the present invention to any specific environment, application or particular method for implementations described in these embodiments. Therefore, the description of these embodiments is for illustrative purposes only and is not intended to limit the present invention. It shall be appreciated that, in the following embodiments and the attached drawings, a part of elements not directly related to the present invention may be omitted from the illustration, and dimensional proportions among individual elements and the numbers of each element in the accompanying drawings are provided only for ease of understanding but not to limit the present invention.

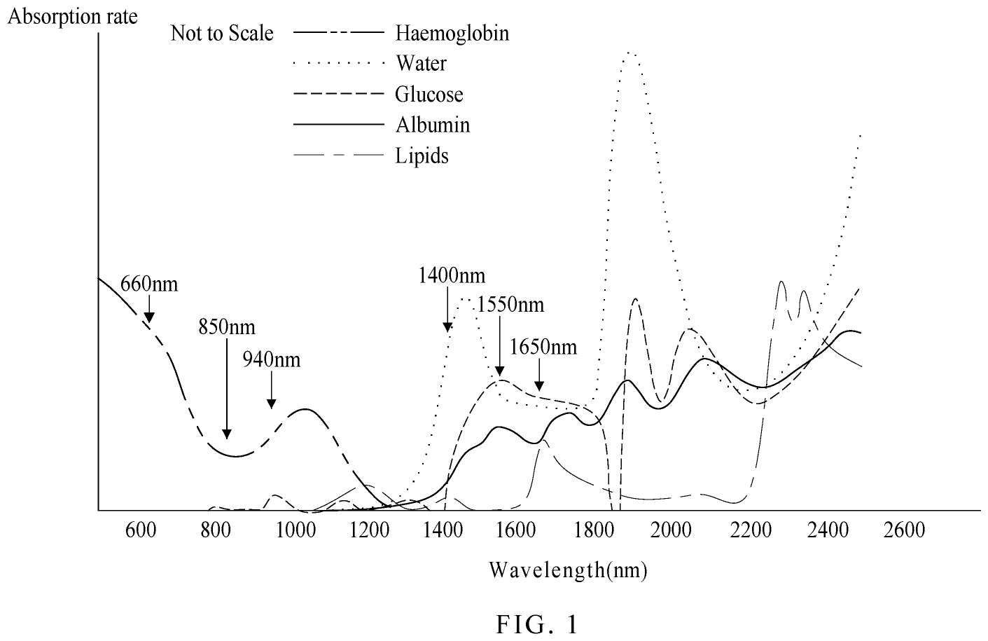

As shown in FIG. 1, based on the theory that infrared light in the wavelength range of 600 nanometers (nm) to 2600 nanometers (nm) can be absorbed and scattered by components such as water, glucose, fat, hemoglobin, and proteins in human tissues for further producing changes in absorption rate, the present invention discloses a physiological value sensing device and its sensing method, particularly a blood glucose concentration sensing device with multi-band optical signals and its sensing algorithm. Specifically, the physiological value sensing device of the present invention is a non-invasive continuous glucose monitoring device (NICGM), which provides the testee with real-time, continuous, and long-term blood glucose concentration monitoring.

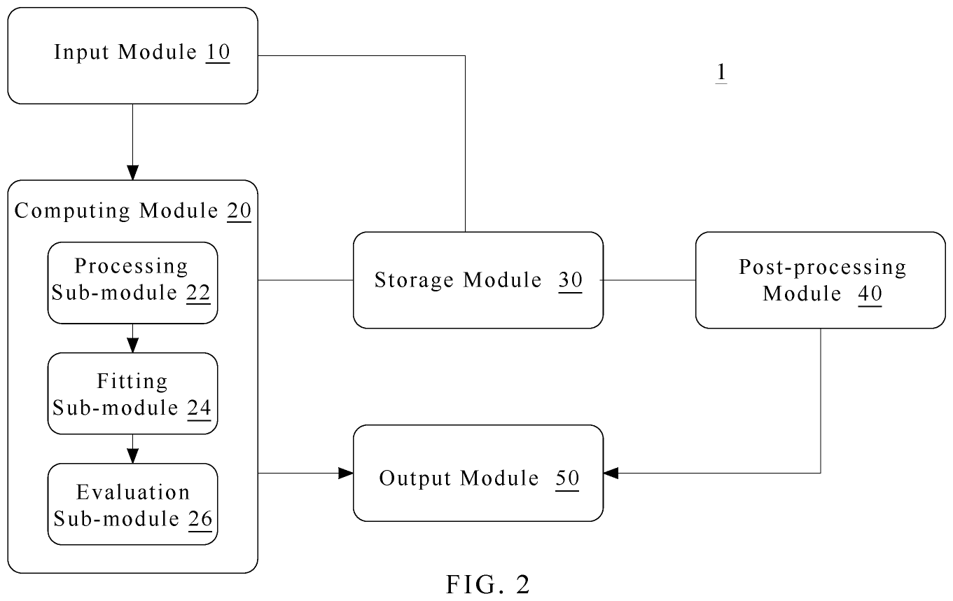

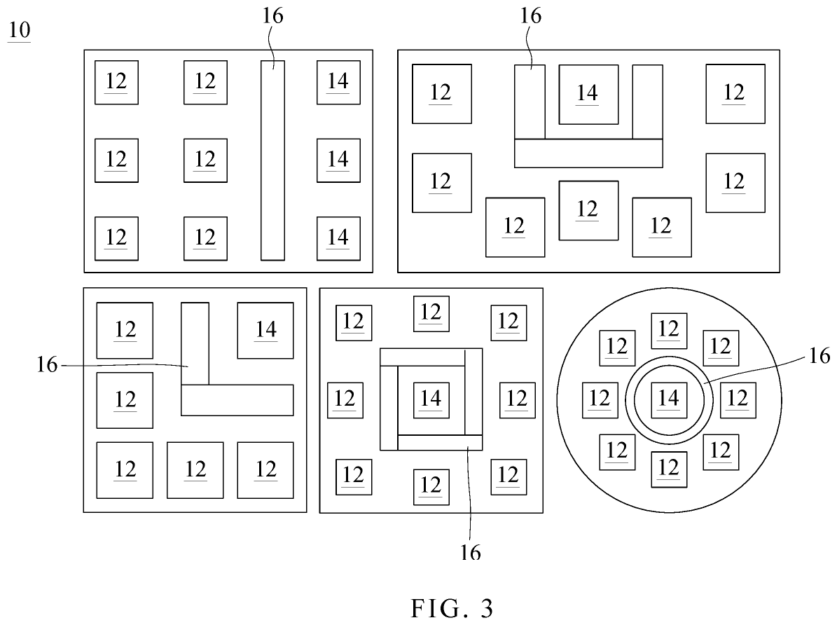

Please refer to FIG. 2, which shows the physiological value sensing device 1 of the present invention. The physiological value sensing device 1 comprises an input module 10, a computing module 20, a storage module 30, a post-processing module 40, and an output module 50. Combining with FIG. 3, it illustrates a top view schematic diagram of several possible embodiments of the input module 10 in the physiological value sensing device 1 of the present invention. The input module 10 includes several light emitting units 12, at least one light detection unit 14, and a microstructure 16. In a preferred embodiment, the light emitting units 12 have three or more light emitting diodes for providing infrared light in different wave bands ranging from 800 nanometers (nm) to 1700 nanometers (nm) and with a wavelength interval of not less than 100 nanometers (nm). Each light emitting diode can adjust the light intensity from 0 to 100%, and the spacing between each light emitting diode is preferably maintained between 1 millimeter (mm) and 2 millimeters (mm).

Furthermore, the light detection unit 14 has one or multiple photodiodes to correspond to multiple light emitting units 12 for receiving the multi-band infrared light emitted by the light emitting units 12. In operation status, the light detection unit 14 receives the infrared light after diffuse reflection from the testee's body, and generates multiple optical signals corresponding to the reflection light. When the physiological value sensing device 1 of the present invention has multiple light detection units 14, the spacing between each light detection unit 14 needs to be fixed, preferably maintained between 1 millimeter (mm) and 2 millimeters (mm). It should be noted that these optical signals generated by the light detection unit 14 after irradiation by infrared light from the corresponding light emitting units 12 contain signals corresponding to various substances in the testee's body, such as, including water, glucose, fat, hemoglobin, and proteins, after infrared light being reflected. Taking the application of the present invention in a non-invasive multi-band blood glucose concentration sensing device as an example, the aforementioned multiple optical signals include several interference signals such as water, fat, hemoglobin, and proteins, as well as a physiological target signal represented by glucose concentration (i.e., blood glucose concentration).

Next, the microstructure 16 is used to separate the light emitting units 12 from the light detection unit 14. In accordance with practical embodiments, the microstructure 16 has an adjustable appearance pattern to maintain the spacing between the microstructure 16 and the light emitting units 12 within a range of 1 millimeter (mm) to 3 millimeters (mm). In the input module 10, multiple spaces can be provided to accommodate multiple light emitting units 12. In a preferred embodiment, the configuration of the microstructure 16 should prevent the light emitted by the light emitting units 12 from being directly received by the light detection unit 14 without being reflected by the testee's body. Therefore, the spatial relationship between the microstructure 16 and the light emitting units 12 should effectively limit the emitting angle of the light emitting units 12 to within a range of 30 degrees for blocking most of the light greater than 30 degrees substantially. Additionally, the microstructure 16, located adjacent to the light detection unit 14, maintains a fixed space between the microstructure 16 and the light detection unit 14. For example, the preferred space therebetween is maintained between 1 millimeter (mm) and 2 millimeters (mm).

To enhance the accuracy of sensing physiological values, one of the features of the physiological value sensing device of the present invention is to create a physiological normal model. This physiological normal model is created by corresponding the optical signals obtained by human tissues under multi-band light irradiation to the multiple physiological sample values of the human tissues at that time. Specifically, when establishing the physiological normal model, various physiological features or characteristics (such as water, glucose, lipids, fats, hemoglobin, and proteins) of the human tissues are first obtained through biochemical instruments. These values are then combined with the optical signals generated by the optical detection of the input module 10 at that time. The computing module 20 calculates and establishes a normal model for making the physiological sample values correspond to the optical signals. This physiological normal model can be used for fitting and evaluation in future tests of testees. Moreover, in a preferred embodiment of the present invention, the light emitting units 12 have multiple light emitting diodes that provide light of different wavelengths and intensities. Additionally, there are different distances between the multiple light-emitting diodes and the light detection unit 14. When establishing the above physiological normal model, by controlling the three variables of wavelength, distance, and intensity, a comprehensive physiological normal model can be established to calculate physiological values close to the real values for improving the sensing accuracy accordingly.

As shown in FIG. 2, the computing module 20 includes a processing sub-module 22, a fitting sub-module 24, and an evaluation sub-module 26. Specifically, the processing sub-module 22 is used to process the optical signals generated by the light detection unit 14 of the input module 10. In particular, it converts the various interference signals (such as feature signals from water, lipids, fats, hemoglobin, protein and the like) contained in the optical signals, as well as the physiological target signal (such as the glucose feature signal), to the physiological normal model. It should be noted that the conversion of the optical signals from the input module to the physiological normal model at this point refers to ensuring that the optical signals obtained from the testee must conform to the data format of the physiological normal model. For example, when establishing the physiological normal model using data formats consisting of three wavelength bands A nanometers (nm), B nm, C nm, distances P millimeters (mm), Q mm, R mm, and light intensities 100%, 90%, . . . , 10%, the subsequent collection of relevant signals by the input module when measuring the optical signals from testees must also correspond to the same data format mentioned above to meet the requirements of normal conversion.

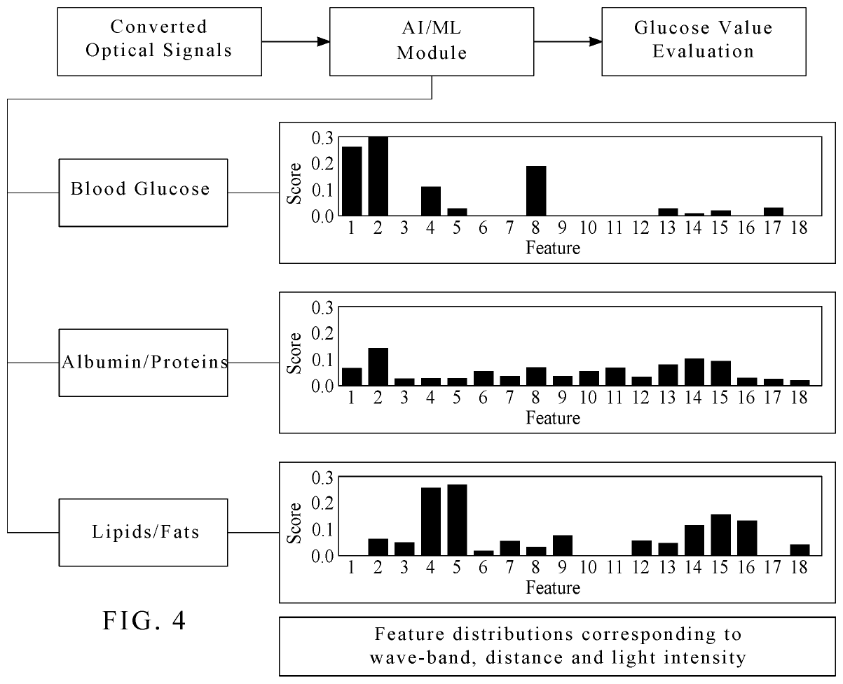

Furthermore, the fitting sub-module 24 is used to fit the converted optical signals with the physiological normal model. Specifically, it fits the various interference signals and the physiological target signal on the optical signals with the physiological normal model to generate a fitting result. Based on the known sample physiological values in the physiological normal model, it eliminates interference signals other than glucose values and generates the physiological target value (such as blood glucose concentration) corresponding to the physiological target signal. Refer to FIG. 4, which illustrates a schematic diagram of the fitting sub-module performing model fitting in an embodiment of the present invention. In this embodiment, the processing sub-module 22 receives several sets of optical signals corresponding to blood glucose, albumin/protein, lipids/fats, and the like collected by the input module. Each type of optical signals contains 18 sets of feature distributions with different wave-bands, distances, and light intensities. These signals are then converted into multiple photoelectric feature values according to the data format of the physiological normal model. The fitting sub-module of the present invention utilizes an artificial intelligence model or machine learning model to fit the converted interference signals (including photoelectric feature values of albumin/protein, lipids/fats, etc.) and the physiological target signal (i.e., the photoelectric feature values of blood glucose) with the known physiological sample values in the physiological normal model to generate a fitting result. Then, the fitting sub-module corrects the interference signals in the optical signals (such as corresponding to photoelectric feature values of albumin/protein, lipids/fats, etc.) and generates the physiological target value (i.e., the photoelectric feature values of blood glucose) corresponding to the physiological target signal. In other words, the estimated physiological values fitted by the fitting sub-module of the present invention are less affected by interfering substances in the body compared to previous techniques. Thus, the present invention can estimate the physiological values of the testee more closely to the real state.

In the following, we will illustrate with an embodiment how the present invention applies artificial intelligence models to fit the converted values and estimate the physiological target values using artificial intelligence/machine learning (AI/ML) models.

As explained earlier, it is supposed the physiological normal model created by the present invention is based on features such as wave-band, distance, and light intensity. And, these three features are further divided into 3 wave-bands (A nanometers (nm), B nm, C nm), 3 intervals between the emitting and detection units (P millimeters (mm), Q mm, R mm), and 10 levels of light intensity (100% (L00), 90% (L90), . . . , 10% (L10)) and the total data format of 90 combinations is created in the physiological normal model. As previously described, when sensing the physiological values of testee, functions for various substances of the testee must be established in the data format of these 90 combinations. For a single substance, at a fixed position, the absorption rate varies with different wave bands. The absorption threshold for different wave bands varies with different levels of light intensity. Additionally, the penetration depth of light into biological tissues varies with different positions and different wave bands. The following is an example, where the function ƒ represents an artificial intelligence model, and the corresponding substance concentration values can be calculated through an optimization process:
Glucose values=ƒ(PAL001, PAL901, . . . , PAL101, PBL001, . . . , RCL101)
Protein values=ƒ(PAL002, PAL902, . . . , PAL102, PBL002, . . . , RCL102)

After actual measurements, the glucose values can be organized into the following matrix format:

| Data Nodes | PAL001 | PAL901 | . . . | PAL101 | PBL001 | . . . | RCL101 |
|---|---|---|---|---|---|---|---|
| 1 | 0.233 | −0.116 | . . . | 0.749 | 1.113 | . . . | 1.367 |
| 2 | 1.001 | 1.015 | . . . | 0.469 | 0.887 | . . . | 0.916 |
| 3 | −0.561 | −1.194 | . . . | 0.256 | 0.981 | . . . | 0.576 |
| . | . | . | [symbol] | . | . | [symbol] | . |
| . | . | . | | . | . | | . |
| . | . | . | | . | . | | . |
| N | 0.323 | 0.176 | . . . | 0.264 | 0.895 | . . . | −0.243 |

In the above data matrix, each of the date in the feature columns are converted by the aforementioned physiological normal model.

The formula for creating the physiological normal model is:

$${x}_{i , std} = \frac{{x}_{i} - \overline{x}}{{s}_{x}}$$

where x̅ is a mean value of the normal model, and sₓ is a standard deviation of the normal model.

Then, optimization is performed on the function ƒ which includes error function optimization and decision making history.
Glucose values cₐ=ƒ(PAL001, PAL901, . . . , PAL101, PBL001, . . . , RCL101)

For each function ƒ, there exists the possibility of error optimization (i.e., minimization). Taking mean square deviation as an example of the error function, it can be represented as:

$$arg min ( \frac{1}{N} {\sum }_{i = 1}^{N} {( {f}_{i} - )}^{2} )$$

where N is the number of samples, ƒᵢ is the actual observed value of the function, and [symbol] is the calculated value of the function model.

If a decision tree type is used as the decision making history, there are two spatial divisions, region 1 and region 2, at each step. For the j-th variable x^{j} with the value s:

$${region}_{1} ( j , s ) = \{ x \vert {x}^{j} \leq s \} \& {region}_{2} ( j , s ) = \{ x \vert {x}^{j} > s \}$$

And solve:

$${min}_{j , s} [ {min}_{{c}_{a 1}} {\sum }_{x ∈ {region}_{1} ( j , s )} {( {c}_{a} - {c}_{a 1} )}^{2} + {min}_{{c}_{a 2}} {\sum }_{x ∈ {region}_{2} ( j , s )} {( {c}_{a} - {c}_{a 2} )}^{2} ]$$

The optimal value s can be found for the j-th variable x^{j}.

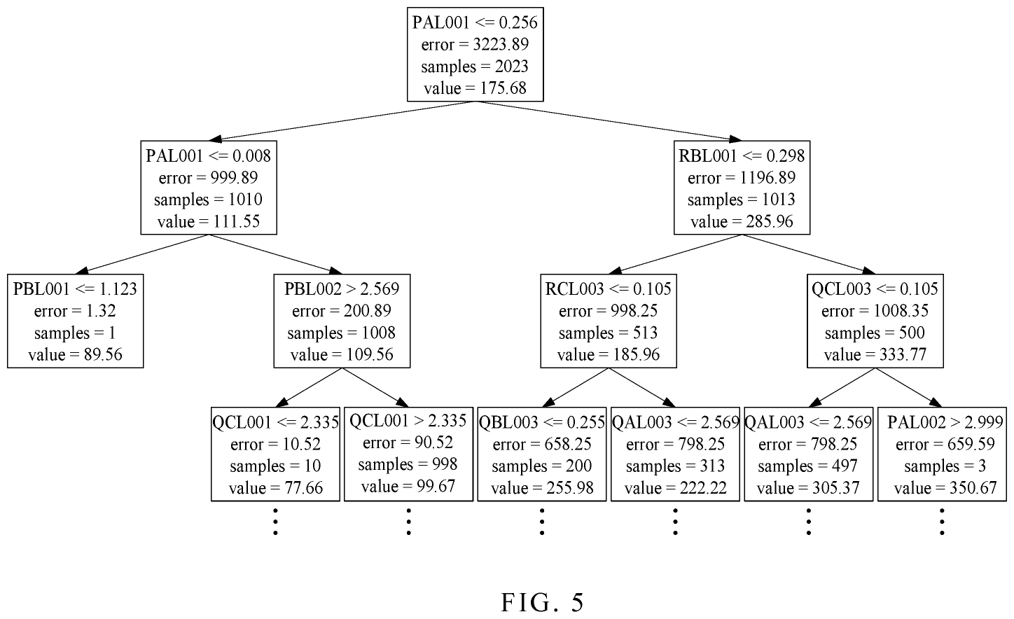

One example of the decision making history of the decision tree can be referred to as shown in FIG. 5. It should be noted that the example shown in FIG. 5 is for illustrative purposes only and does not represent actual scenarios. For example, if the feature value of PAL001 (glucose feature item: interval distance between the light emitting unit and the light detection unit is P, the wave band is A, and the light intensity L is 100%) calculated by the algorithm mentioned above is less than or equal to 0.256, the decision tree will proceed to the left side. Conversely, if the feature value of PAL001 is greater than 0.256, the decision tree will proceed to the right side. Then, continue to evaluate the next feature value. If the feature value of PBL001 is less than or equal to 1.123, according to the decision tree, the estimated blood glucose concentration at this time will be 89.56 milligrams per deciliter (mg/dL).

Furthermore, another feature of the present invention is that the computing module 20 further includes an evaluation sub-module 26, which is used to analyze the model based on the fitting result obtained from the fitting sub-module 24 and output a feature importance evaluation, such as, but not limited to, the SHAP (SHapley Additive explanations) feature importance evaluation. This evaluation considers the contribution of each feature to the prediction results and assigns a SHAP value to each feature. These values describe the impact of each feature on the model's predictions with positive values indicating an increase in the predicted values and negative values indicating a decrease in the predicted values. SHAP values can be used to explain how artificial intelligence models use various optical features to predict blood glucose concentration. These optical features may include physiological values, environmental factors, and the like. Using SHAP values, the impact of each feature on the output of the artificial intelligence model can be evaluated for providing a better understanding of the prediction process of the artificial intelligence model accordingly.

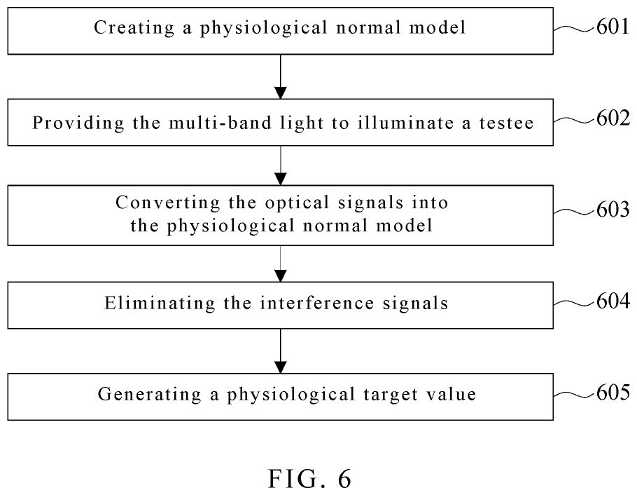

Based on the abovementioned, please refer to FIG. 6 which discloses a physiological value sensing method of the present invention. The physiological value sensing method includes the following steps. In step 601, first, create a physiological normal model which has multiple physiological sample values corresponding to a multi-band light. In step 602, provide the multi-band light to illuminate a testee for generating multiple optical signals, wherein these optical signals include a plurality of interference signals and a physiological target signal. In step 603, convert these interference signals and the physiological target signal to the physiological normal model. In step 604, eliminate these interference signals based on the physiological sample values. In step 605, generate a physiological target value corresponding to the physiological target signal. Specifically, taking the measurement of blood glucose concentration as an example, the blood glucose sensing method of the present invention can correct the influence of other interfering substances in testee's body on the measurement of blood glucose concentration. Mainly, it uses the input module to conduct measurements on the testee to generate a plurality of optical signals for evaluating blood glucose and eliminating interference substances. The computing module obtains several uncorrected feature values related to the testees corresponding to the optical signals, including a set of blood substance concentration values, such as blood glucose concentration value, protein concentration value, lipid concentration value, water influence value, and other substance influence values. Finally, using the calculation of the previously established physiological normal model, a corrected blood glucose concentration value is generated as the testee's evaluation of the real blood glucose concentration.

Additionally, as shown in FIG. 2, the storage module 30 of the physiological value sensing device 1 of the present invention is used to store the optical signals generated by the input module 10, the intermediate signals such as the converted interference signals and the converted physiological target signal after conversion by the computing module 20, the physiological target values, and the feature importance evaluation, as well as the final results. Furthermore, the post-processing module 40 of the physiological value sensing device 1 is used to calculate the values including the optical signals stored in the storage module 30 to establish a long-term trend report after the system has operated for a period of time. The output module 50 of the physiological value sensing device 1 of the present invention is used to output the physiological target values, the feature importance evaluation, and the long-term trend report, and can output the values of the computing module as well as image files for display on a monitor.

The physiological value sensing device and its sensing method of the present invention have the advantages of non-invasion, multi-band light, continuous long-term monitoring, real-time offline detection of physiological values, and so on. Compared to conventional techniques, the present invention can solve (1) the pain caused to the testee by invasive sensing devices or methods; (2) the possibility of inflammation at the site of skin invasion due to long-term wearing for continuous monitoring of physiological values and causing psychological concerns and additional burden on the testees, caregivers, or the healthcare system; and (3) multiple specific interference factors for the physiological values in human tissues which can be excluded with the sensing device and associated sensing algorithms of the present invention so relevant values can be corrected to provide physiological values close to reality.

The above embodiments are used only to illustrate the implementations of the present invention and to explain the technical features of the present invention, and are not used to limit the scope of the present invention. Any modifications or equivalent arrangements that can be easily accomplished by people skilled in the art are considered to fall within the scope of the present invention, and the scope of the present invention should be limited by the claims of the patent application.

What is claimed is:

1. A physiological value sensing device, comprising:
an input module for providing a multi-band light to illuminate a testee to generate a plurality of optical signals wherein the optical signals include a plurality of interference signals and a physiological target signal; and
a computing module for establishing a physiological normal model having multiple physiological sample values corresponding to the multi-band light, converting the interference signals and the physiological target signal into the physiological normal model for fitting, wherein a physiological target value corresponding to the physiological target signal is generated after eliminating the interference signals based on the physiological sample values.

2. The physiological value sensing device of claim 1, wherein the input module includes a plurality of light emitting units and at least one light detection unit, wherein the light emitting units are used to provide the multi-band light and are capable of adjusting the light intensity of the multi-band light, the at least one light detection unit is used to receive a reflection light reflected by the multi-band light illuminating the testee, and generate the optical signals corresponding to the reflection light.

3. The physiological value sensing device of claim 2, wherein the light emitting units are capable of providing the multi-band light with a wavelength of 800 nanometers to 1700 nanometers and a wavelength interval of not less than 100 nanometers.

4. The physiological value sensing device of claim 2, wherein the input module further includes a microstructure, disposed adjacent to the at least one light detection unit to prevent the multi-band light provided by the light emitting units from being received by the at least one light detection unit without being reflected by the testee.

5. The physiological value sensing device of claim 1, wherein the computing module includes a processing sub-module, a fitting sub-module and an evaluation sub-module,
wherein the processing sub-module is used to process and convert the interference signals and the physiological target signal into the physiological normal model,
the fitting sub-module is used to fit the converted interference signals and the converted physiological target signal with the physiological normal model to generate a fitting result, and eliminate the converted interference signals based on the physiological sample values and generate the physiological target value corresponding to the converted physiological target signal, and
the evaluation sub-module is used to output a feature importance evaluation corresponding to the fitting result.

6. The physiological value sensing device of claim 5, further comprising a storage module for storing the optical signals generated by the input module, the converted interference signals and the converted physiological target signal, the physiological target value and the feature importance evaluation.

7. The physiological value sensing device of claim 6, further comprising a post processing module for creating a long-term trend report corresponding to the optical signals.

8. The physiological value sensing device of claim 7, further comprising an output module for outputting the physiological target value and the feature importance evaluation and the long-term trend report.

9. A physiological value sensing method, comprising:
creating a physiological normal model wherein the physiological normal model has multiple physiological sample values corresponding to a multi-band light;
providing the multi-band light to illuminate a testee to generate a plurality of optical signals wherein the optical signals include a plurality of interference signals and a physiological target signal;
converting the interference signals and the physiological target signal into the physiological normal model for fitting;
eliminating the interference signals based on the physiological sample values; and
generating a physiological target value corresponding to the physiological target signal.

10. The physiological value sensing method of claim 9, wherein the step of providing the multi-band light is to provide the multi-band light with a wavelength of 800 nanometers to 1700 nanometers and a wavelength interval of not less than 100 nanometers.

11. The physiological value sensing method of claim 9, further comprising a step of providing an artificial intelligence model for fitting the converted interference signals and the converted physiological target signal with the physiological sample values of the physiological normal model to generate a fitting result, and generating the physiological target value corresponding to the converted physiological target signal after eliminate the converted interference signals based on the fitting result.
